(19) United States
(12) Patent Application Publication
(10) Pub. No.: US 2022/0096871 A1
(43) Pub. Date: Mar. 31, 2022

(54) ROTATING GANTRY AND RADIOTHERAPY EQUIPMENT

(71) Applicants: OUR INNOBEAM MEDICAL CO., LTD, Beijing (CN); OUR UNITED CORPORATION, Xi'an City (CN)

(72) Inventor: Zhao GUO, Xi'an City (CN)

(21) Appl. No.: 17/489,061

(22) Filed: Sep. 29, 2021

(30) Foreign Application Priority Data
Sep. 30, 2020 (CN) 202022218774.5

(51) Int. Cl. A61N 5/10

(52) U.S. Cl. CPC A61N 5/1081; A61N 5/1048

(57) ABSTRACT

The present disclosure discloses a rotating gantry and radiotherapy equipment, and belongs to the technical filed of medical instruments. The rotating gantry can include a frame-shaped support, an annular rotating frame, a drive assembly, and a ring gear disk. The annular rotating frame in the rotating gantry can be disposed on one side of the frame-shaped support; and the ring gear disk and the drive assembly can be disposed on the other side of the frame-shaped support. The drive assembly can be configured to drive, via the ring gear disk, the annular rotating frame to rotate.

### CROSS-REFERENCE TO RELATED APPLICATION

This application is based on and claims priority to the Chinese Patent Application No. 202022218774.5, filed on Sep. 30, 2020 and entitled “ROTATING GANTRY AND RADIOTHERAPY EQUIPMENT,” the disclosure of which is herein incorporated by reference in its entirety.

### TECHNICAL FIELD

The present disclosure relates to the technical filed of medical instruments, and in particular, to a rotating gantry and radiotherapy equipment.

### BACKGROUND

Radiotherapy is an important technique for cancer treatment. Radiotherapy equipment is key medical equipment to carry out radiotherapy. The radiotherapy equipment usually includes a C-arm rotating gantry and a treatment head fixedly connected to the rotating gantry.

### SUMMARY

Embodiments of the present disclosure provide a rotating gantry and radiotherapy equipment.

According to one aspect of the embodiments of the present disclosure, a rotating gantry is provided. The rotating gantry includes.

a frame-shaped support;

an annular rotating frame disposed on one side of the frame-shaped support and configured to support a target device:

a ring gear disk disposed on the other side of the frame-shaped support and fixedly connected to the annular rotating frame; and

a drive assembly including a drive motor and a drive gear, wherein the drive motor is connected to the drive gear, the drive gear is meshed with the ring gear disk, and under driving of the drive motor, the drive gear is configured to drive the ring gear disk and the annular rotating frame to rotate relative to the frame-shaped support, such that the target device is driven to rotate.

In some embodiments, the drive motor is provided with a transmission shaft, wherein the drive gear is sleeved on the transmission shaft and is fixedly connected to the transmission shaft.

In some embodiments, the drive motor is a torque motor.

In some embodiments, the drive assembly further includes a mounting base, wherein the mounting base is fixedly connected to one side, distal from the annular rotating frame, of the frame-shaped support, and is fixedly connected to the drive motor and movably connected to the drive gear.

In some embodiments, an axis of the mounting base is not coincident with an axis of the drive gear.

In some embodiments, the mounting base includes a base body and a positioning member, wherein the positioning member is fixedly connected to one side of the base body, the other side of the base body is fixedly connected to the drive motor, and the base body is movably connected to the drive gear;

one side, distal from the annular rotating frame, of the frame-shaped support is provided with a positioning hole, wherein the positioning hole is matched with the positioning member in shape, and in the case that the positioning member is disposed in the positioning hole and the drive gear is meshed with the ring gear disk, one side, distal from the drive motor, of the base body is fixedly connected to one side, distal from the annular rotating frame, of the frame-shaped support.

In some embodiments, the base body is provided with a receiving chamber, wherein an outer side surface of the base body is provided with an opening communicated with the receiving chamber, the drive gear is disposed in the receiving chamber, and the drive gear is meshed with the ring gear disk through the opening; and

the mounting base further includes a support bearing disposed between the positioning member and the transmission shaft, wherein the positioning member is movably connected to the transmission shaft via the support bearing.

In some embodiments, the drive assembly further includes a right-angle reducer fixedly connected to the frame-shaped support, wherein the right-angle reducer is disposed between the drive motor and the drive gear, and the drive motor is connected to the drive gear via the right-angle reducer.

In some embodiments, the drive motor is provided with a transmission shaft, wherein the transmission shaft is connected to one end of the right-angle reducer, the other end of the right-angle reducer is connected to the drive gear, and an axis of the transmission shaft is perpendicular to an axis of the drive gear.

In some embodiments, the rotating gantry further includes a rotary assembly, wherein the rotary assembly is disposed on the frame-shaped support between the annular rotating frame and the ring gear disk, and the ring gear disk and the annular rotating frame are movably connected to the frame-shaped support via the rotary assembly.

In some embodiments, the rotary assembly further includes an inner ring body and an outer ring body movably connected to the inner ring body, wherein the outer ring body is fixedly connected to the frame-shaped support, and the inner ring body is fixedly connected to the annular rotating frame and the ring gear disk.

In some embodiments, the rotary assembly further includes a plurality of spheres disposed between the inner ring body and the outer ring body, wherein the inner ring body is movably connected to the outer ring body via the plurality of spheres.

In some embodiments, the annular rotating frame is disk-shaped, and the target device is fixedly connected to one side, distal from the frame-shaped support, of the disk-shaped annular rotating frame.

In some embodiments, the annular rotating frame is roller-shaped, and the target device is fixedly connected to the inner wall of the roller-shaped annular rotating frame.

In some embodiments, the ring gear disk and the drive gear are both an external gear.

According to another aspect of the embodiments of the present disclosure, radiotherapy equipment is provided. The radiotherapy equipment includes a rotating gantry and a target device, wherein the rotating gantry includes:

a frame-shaped support:

an annular rotating frame disposed on one side of the frame-shaped support and connected to the target device:

a ring gear disk disposed on the other side of the frame-shaped support and fixedly connected to the annular rotating frame; and

a drive assembly including a drive motor and a drive gear, wherein the drive motor is connected to the drive gear, the drive gear is meshed with the ring gear disk, and under driving of the drive motor, the drive gear is configured to drive the ring gear disk and the annular rotating frame to rotate relative to the frame-shaped support, such that the target device is driven to rotate.

In some embodiments, the target device includes a treatment head and a shield that are arranged oppositely; and the radiotherapy equipment is provided with a treatment area, wherein the treatment area is disposed between the treatment head and the shield.

In some embodiments, the target device further includes an imaging assembly and a detector that are arranged oppositely, wherein the treatment area is disposed between the imaging assembly and the detector, and a direction of a radiation beam emitted by the imaging assembly is intersected with a direction of a radiation beam emitted by the treatment head.

In some embodiments, the radiotherapy equipment further includes a treatment couch, wherein the treatment couch is disposed on one side, proximal to the annular rotating frame, of the rotating gantry.

In some embodiments, the treatment couch includes a treatment couch body and a foldable support frame fixedly connected to the treatment couch body, wherein the foldable support frame is configured to drive the treatment couch body to move in a height direction of the radiotherapy equipment.

### BRIEF DESCRIPTION OF THE DRAWINGS

For clear descriptions of the technical solutions in the embodiments of the present disclosure, the following briefly introduces the accompanying drawings required for describing the embodiments. Apparently, the accompanying drawings in the following description show merely some embodiments of the present disclosure, and a person of ordinary skill in the art may still derive other drawings from these accompanying drawings without creative efforts.

FIG. 1 is a schematic structural view of a rotating gantry according to the present disclosure;

FIG. 2 is a side view of the rotating gantry shown in FIG. 1;

FIG. 3 is a schematic structural view of a drive assembly according to an embodiment of the present disclosure;

FIG. 4 is a schematic structural view of another rotating gantry according to an embodiment of the present disclosure;

FIG. 5 is a schematic structural view of a rotating gantry according to another embodiment of the present disclosure;

FIG. 6 is a half cross-sectional schematic view of the rotating gantry shown in FIG. 4;

FIG. 7 is a schematic structural view of a drive assembly according to an embodiment of the present disclosure;

FIG. 8 is a sectional view of the drive assembly shown in FIG. 7;

FIG. 9 is a schematic view of a location relationship between a drive gear and a ring gear disk before adjustment for a mounting base according to an embodiment of the present disclosure;

FIG. 10 is a schematic view of a location relationship between a drive gear and a ring gear disk after adjustment for a mounting base according to an embodiment of the present disclosure:

FIG. 11 is a schematic structural view of another rotating gantry according to another embodiment of the present disclosure;

FIG. 12 is a schematic structural view of still another rotating gantry according to another embodiment of the present disclosure;

FIG. 13 is a schematic view of a plane on one side of the rotating gantry shown in FIG. 12;

FIG. 14 is a schematic view of a plane on the other side of the rotating gantry shown in FIG. 12;

FIG. 15 is a schematic structural view of a right-angle reducer according to an embodiment of the present disclosure;

FIG. 16 is a schematic structural view of radiotherapy equipment according to an embodiment of the present disclosure, and

FIG. 17 is a schematic structural view of another piece of radiotherapy equipment according to an embodiment of the present disclosure.

### DETAILED DESCRIPTION

For clearer descriptions of the objectives, technical solutions, and advantages of the present disclosure, embodiments of the present disclosure are described in detail hereinafter with reference to the accompanying drawings.

In related art, a size of a C-arm rotating gantry in radiotherapy equipment is usually large. It is difficult to transport a rotating gantry with a large size. In addition, a support base for supporting the rotating gantry needs to be pre-embedded in the ground, to effectively support the rotating gantry with the large size. As a result, the rotating gantry becomes more difficult to mount.

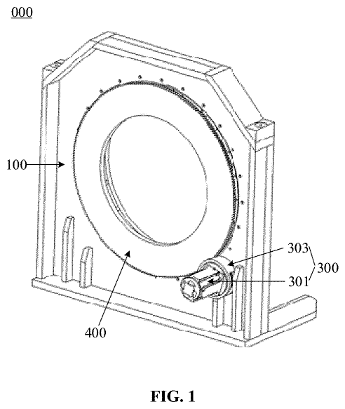

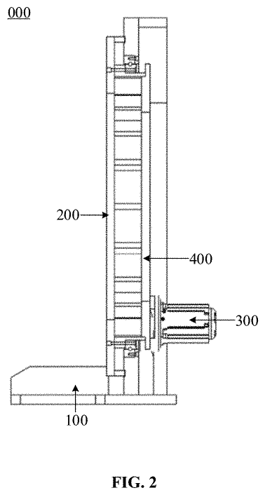

Referring to FIG. 1 and FIG. 2, FIG. 1 is a schematic structural view of a rotating gantry according to the present disclosure; and FIG. 2 is a side view of the rotating gantry shown in FIG. 1. The rotating gantry 000 may include:

a frame-shaped support 100, an annular rotating frame 200, a drive assembly 300, and a ring gear disk 400.

The annular rotating frame 200 may be disposed on one side of the frame-shaped support 100 and configured to support a target device. For example, the target device may include a treatment head, a shield, an imaging assembly, a detector, and the like.

The ring gear disk 400 may be disposed on the other side of the frame-shaped support 100. The ring gear disk 400 may be fixedly connected to the annular rotating frame 200. In this embodiment of the present disclosure, the annular rotating frame 200 and the ring gear disk 400 may be respectively disposed on two opposite sides of the frame-shaped support 100. Both the annular rotating frame 200 and the ring gear disk 400 may be movably connected to the frame-shaped support 100. In this way, in the case that the ring gear disk 400 is rotating relative to the frame-shaped support 100, the ring gear disk 400 is configured to drive the annular rotating frame 200 to rotate together.

It should be noted that, the annular rotating frame 200 is usually provided with a first inner ring. The frame-shaped support 100 may be provided with a second inner ring communicated with the first inner ring. The ring gear disk 400 may be provided with a third inner ring communicated with the second inner ring. In this case, an area, which a treatment couch in radiotherapy equipment can enter, can be formed in the rotating gantry 000 through the first inner ring, the second inner ring, and the third inner ring that are communicated with one another, such that any position of a patient on the treatment couch can be treated by the radiotherapy equipment.

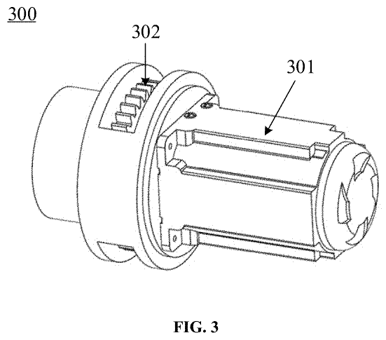

To see the structure of the drive assembly 300 more clearly, refer to FIG. 3, which is a schematic structural view of a drive assembly according to an embodiment of the present disclosure. The drive assembly 300 may include a drive motor 301 and a drive gear 302. The drive motor 301 may be connected to the drive gear 302. The drive gear 302 may be meshed with the ring gear disk 400. In this way, the drive gear 302 can rotate under driving of the drive motor 301. Then the rotating drive gear 302 is configured to drive the ring gear disk 400 and the annular rotating frame 200 to rotate relative to the frame-shaped support 100, such that the target device is driven to rotate.

In some embodiments, the ring gear disk 400 and the drive gear 302 may be both an external gear. In this case, the stability of the rotating of the ring gear disk 400 driven by the drive gear 302 is improved; and the meshing process between the drive gear 302 and the ring gear disk 400 can be simplified.

In this embodiment of the present disclosure, the annular rotating frame 200 in the rotating gantry 000 is disposed on one side of the frame-shaped support 100, and the ring gear disk 400 and the drive gear 302 in the drive assembly 300 are both disposed on the other side of the frame-shaped support 100. In this way, components in the rotating gantry 000 are arranged closely. In addition, the frame-shaped support 100 takes the shape of a flat plate. Therefore, the size of the rotating gantry 000 are relatively small, which effectively reduces the volume of the rotating gantry 000.

In this way, the transport difficulty of the rotating gantry 000 can be reduced. Due to a lower transport difficulty of the rotating gantry 000, it is more efficient to replace a rotating gantry of radiotherapy equipment in an old machine room in a hospital with the rotating gantry 000.

In addition, the frame-shaped support 100 can be directly disposed on the ground to support the rotating annular rotating frame 200, with no need to pre-embed the frame-shaped support 100 in the ground. This effectively reduces the mounting difficulty of the rotating gantry 000. In the case that the frame-shaped support 100 can be directly disposed on the ground, there is no need to pre-set pits on the ground of a machine room in a hospital, which effectively reduces the construction difficulty of the machine room.

In summary, the rotating gantry in this embodiment of the present disclosure includes a frame-shaped support, an annular rotating frame, a drive assembly, and a ring gear disk. The drive assembly may be configured to drive, via the ring gear disk, the annular rotating frame to rotate. The annular rotating frame in the rotating gantry is disposed on one side of the frame-shaped support; and the ring gear disk and the drive assembly are disposed on the other side of the frame-shaped support. In this case, components in the rotating gantry are arranged closely. In addition, the frame-shaped support takes the shape of a flat plate. Therefore, the size of the rotating gantry is relatively small, which effectively reduces the volume of the rotating gantry. In this way, the transport difficulty of the rotating gantry can be effectively reduced. In addition, the frame-shaped support can be directly disposed on the ground, instead of being pre-embedded in the ground, which effectively lowers the mounting difficulty of the rotating gantry.

In this embodiment of the present disclosure, the annular rotating frame 200 in the rotating gantry 000 has a plurality of shapes. The embodiment of the present disclosure is illustratively described by using the following two optional embodiments as examples.

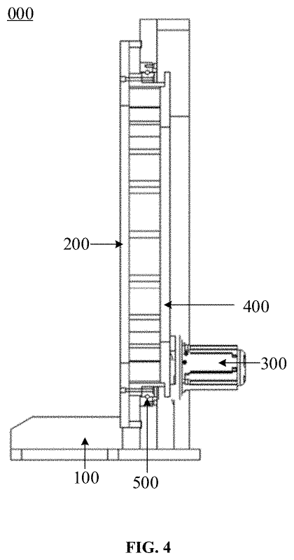

In a first optional embodiment, FIG. 4 is a schematic structural view of another rotating gantry according to an embodiment of the present disclosure. The annular rotating frame 200 in the rotating gantry 000 may be disk-shaped. In this case, the target device needs to be fixedly connected to an end surface on one side, distal from the frame-shaped support 100, of the annular rotating frame 200.

It should be noted that, in the case that the annular rotating frame 200 is disk-shaped, the annular rotating frame 200 takes the shape of a flat plate. In this way, the size of the rotating gantry 000 can be further reduced, thereby reducing the volume of the rotating gantry 000.

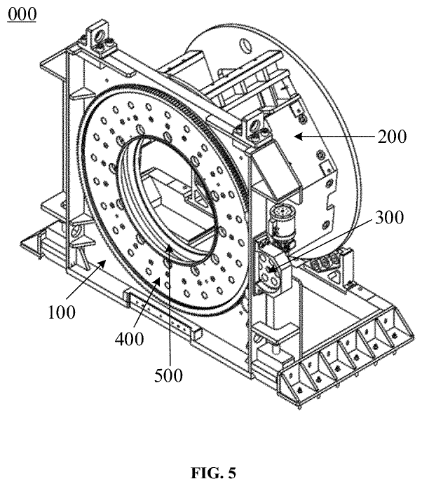

In a second optional embodiment, referring to FIG. 5, which is a schematic structural view of a rotating gantry according to another embodiment of the present disclosure, the annular rotating frame 200 in the rotating gantry 000 may be roller-shaped. In this case, the target device needs to be fixedly connected to an inner wall of the annular rotating frame 200.

In some embodiments, as shown in FIG. 4 and FIG. 5, the rotating gantry 000 may further include a rotary assembly 500. The rotary assembly 500 is disposed on the frame-shaped support 100 between the ring gear disk 400 and the annular rotating frame 200. The ring gear disk 400 and the annular rotating frame 200 may be movably connected to the frame-shaped support 100 via the rotary assembly 500.

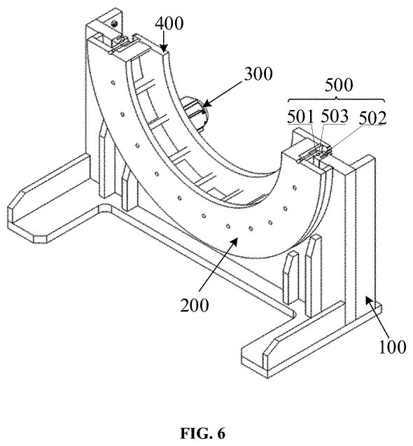

The following embodiments illustratively describe the structure of the rotary assembly 500 in the rotating gantry 000 by taking that the annular rotating frame 200 is disk-shaped as an example. In an exemplary embodiment, FIG. 6 is a half cross-sectional schematic view of the rotating gantry shown in FIG. 4. The rotary assembly 500 may include an inner ring body 501 and an outer ring body 502 movably connected to the inner ring body 501. For example, the rotary assembly 500 may include a plurality of spheres 503 disposed between the inner ring body 501 and the outer ring body 502. The inner ring body 501 may be movably connected to the outer ring body 502 via the plurality of spheres 503, and can rotate relative to the outer ring body 502. It should be noted that, the spheres 503 disposed between the inner ring body 501 and the outer ring body 502 are also generally referred to as balls.

The outer ring body 502 of the rotary assembly 500 may be fixedly connected to the frame-shaped support 100. For example, the outer ring body may be fixedly connected to the frame-shaped support 100 via a screw. The inner ring body 501 in the rotary assembly 500 may be fixedly connected to the annular rotating frame 200 and the ring gear disk 400 separately. For example, the inner ring body 501 may be fixedly connected to the annular rotating frame 200 and the ring gear disk 400 via screws separately. In this way, the annular rotating frame 200 and the ring gear disk 400 can rotate on the frame-shaped support 100 together.

In this embodiment of the present disclosure, there are a plurality of fashions for mounting the drive assembly 300 in the rotating gantry 000 to the frame-shaped support 100. The embodiment of the present disclosure is illustratively described by taking the following two optional embodiments as examples.

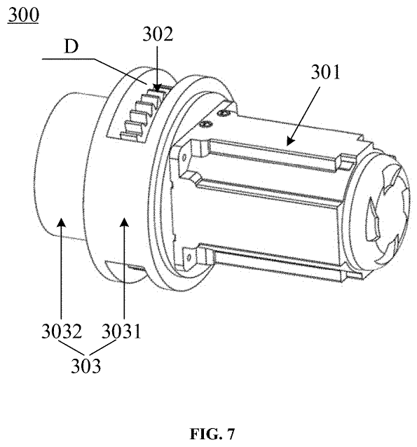

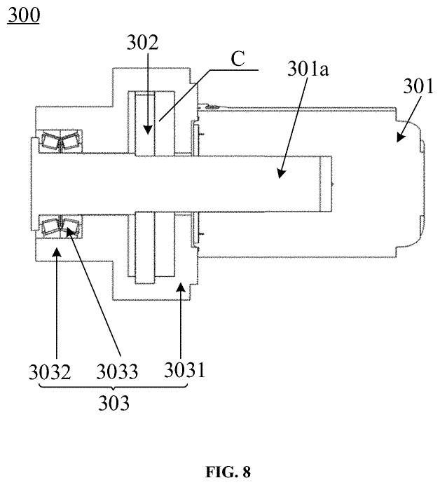

In a first optional embodiment, referring to FIG. 1, FIG. 7, and FIG. 8, FIG. 1 is a schematic structural view of a rotating gantry according to the present disclosure. FIG. 7 is a schematic structural view of a drive assembly according to an embodiment of the present disclosure, and FIG. 8 is a sectional view of the drive assembly shown in FIG. 7. The drive motor 301 in the drive assembly 300 is provided with a transmission shaft 301a. The drive gear 302 in the drive assembly 300 may be directly sleeved on the transmission shaft 301a of the drive motor 301, and may be fixedly connected to the transmission shaft 301a of the drive motor 301. In an exemplary embodiment, the drive gear 302 may be fixedly connected to the transmission shaft 301a via keyed connection or welding. This is not limited in this embodiment of the present disclosure.

The transmission shaft 301a in the drive motor 301 can rotate in the case that the drive motor 301 works, such that the drive gear 302 sleeved on the transmission shaft 301a can rotate, therefore the ring gear disk 400 and the annular rotating frame 200 can be driven, by the drive gear 302, to rotate relative to the frame-shaped support 100.

In this case, the drive motor 301 may be a torque motor. A torque motor has characteristics of low revolution speed and large torque. Therefore, the torque motor can be directly configured to drive, via the drive gear 302 and the ring gear disk 400, the annular rotating frame 200 to rotate, without disposing complex transmission structures such as a reducer and a commutator between the drive motor 301 and the drive gear 302, such that the structure of the drive assembly 300 is effectively simplified.

In some embodiments, the drive assembly 300 in the rotating gantry 000 may further include a mounting base 303. The mounting base 303 may be fixedly connected to one side, distal from the annular rotating frame 200, of the frame-shaped support 100. The mounting base 303 may be fixedly connected to the drive motor 301 and movably connected to the drive gear 302. In the drive assembly 300, the mounting base 303 can be configured to support the drive motor 301 and the drive gear 302; and the drive motor 301 and the drive gear 302 can be mounted on the frame-shaped support 100 via the mounting base 303.

In this embodiment of the present disclosure, an axis of the mounting base 303 in the drive assembly 300 is not coincident with an axis of the transmission shaft 301a in the drive motor 301. In other words, the axis of the mounting base 303 is not coincident with the axis of the drive gear 302 in the drive motor 301. In this case, when mounting the drive assembly 300, the position of the drive gear 302 can be adjusted by rotating the mounting base 303, such that the drive gear 302 and the ring gear disk 400 can be successfully meshed with each other; and the meshing gap between the drive gear 302 and the ring gear disk 400 can also be adjusted by rotating the mounting base 303. Therefore, the meshing precision between the drive gear 302 and the ring gear disk 400 can be effectively improved, and the driving precision of subsequent driving can be further improved. In response to the meshing precision between the drive gear 302 and the ring gear disk 400 being adjusted by rotating the mounting base 303, the mounting base 303 can be fixed on one side, distal from the annular rotating frame 200, of the frame-shaped support 100.

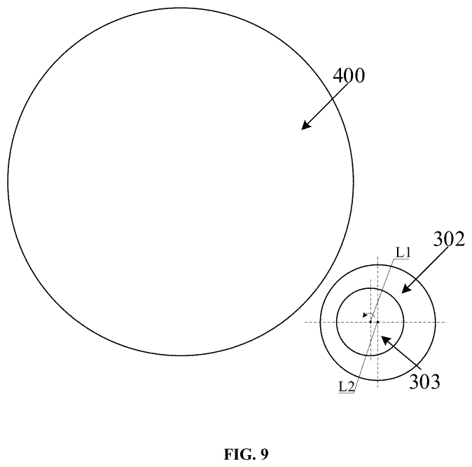

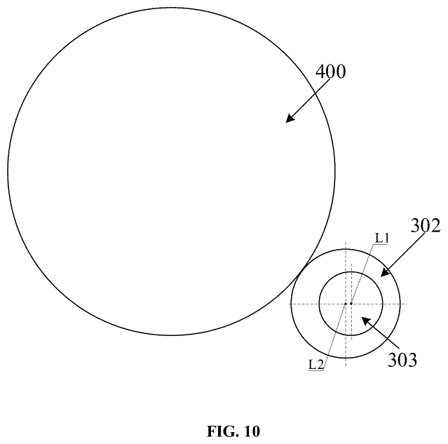

For example, referring to FIG. 9 and FIG. 10, FIG. 9 is a schematic view of a location relationship between a drive gear and a ring gear disk before adjustment of a mounting base according to an embodiment of the present disclosure, and FIG. 10 is a schematic view of a location relationship between a drive gear and a ring gear disk after adjustment of a mounting base according to an embodiment of the present disclosure. The axis L1 of the mounting base 303 is not coincident with the axis L2 of the drive gear 302. Therefore, in response to rotating the mounting base 303, the drive gear 302 can rotate around the axis L1 of the mounting base 303, such that the drive gear 302 and the ring gear disk 400 can be successfully meshed with each other. In addition, the meshing gap between the drive gear 302 and the ring gear disk 400 can also be adjusted.

In this embodiment of the present disclosure, as shown in FIG. 7 and FIG. 8, the mounting base 303 in the drive assembly 300 may include a base body 3031 and a positioning member 3032 fixedly connected to one side of the base body 3031. The other side of the base body 3031 may be fixedly connected to the drive motor 301. The base body 3031 may be movably connected to the drive gear 302.

In an exemplary embodiment, the base body 3031 is provided with a receiving chamber C, and an outer side surface of the base body 3031 is provided with an opening D communicated with the receiving chamber C. The drive gear 302 may be disposed in the receiving chamber C of the base body 3031, and movably connected to the base body 3031. External teeth of the drive gear 302 can be exposed out of the base body 3031 through the opening D of the base body 3031, such that the drive gear 302 can be meshed with the ring gear disk 400 through the opening D of the base body 3031. In the present disclosure, the transmission shaft 301a in the drive motor 301 may be movably connected to the positioning member 3032 in the mounting base 303. For example, the mounting base 303 may further include a support bearing 3033. The positioning member 3032 may be movably connected to the transmission shaft 301a via the support bearing 3033. In this way, movable connection between the positioning member 3032 and the transmission shaft 301a can be achieved via the support bearing 3033, such that the drive gear 302 sleeved on the transmission shaft 301a can be disposed in the receiving chamber C of the base body 3031.

In this embodiment of the present disclosure, the frame-shaped support 100 is provided with a positioning hole matched with the positioning member 3032 in the mounting base 303 in shape. In an exemplary embodiment, the positioning member 3032 may be cylindrical; and the positioning hole may be a circular hole matched with the positioning member 3032 in shape. Therefore, in the case that the positioning member 3032 is disposed in the positioning hole, the positioning member 3032 can be rotated in the positioning hole, to adjust the position of the drive gear 302. In response to the drive gear 302 being meshed with the ring gear disk 400, one side, distal from the drive motor 301, of the base body 3031 may be fixedly connected to one side, distal from the annular rotating frame 200, of the frame-shaped support 100. For example, the side, distal from the drive motor 301, of the base body 3031 may be fixed to the frame-shaped support 100 via a fixing member such as a screw.

In the case that the drive assembly 300 in the rotating gantry 000 needs to be mounted on the frame-shaped support 100, first, the positioning member 3032 in the mounting base 303 may be mounted in the positioning hole of the frame-shaped support 100, to primarily assemble the drive assembly 300 on the frame-shaped support 100; then, the drive gear 302 is meshed with the ring gear disk 400 by rotating the mounting base 303, and the meshing gap between the drive gear 302 and the ring gear disk 400 is guaranteed; and finally, the side, distal from drive motor 301, of the base body 3031 in the mounting base 303 may be fixed on the frame-shaped support 100 via a plurality of fasteners, thereby mounting the drive assembly 300 on the frame-shaped support 100.

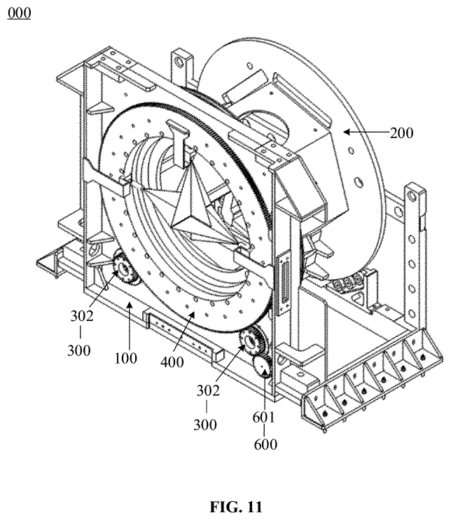

It should be noted that, FIG. 1 is illustrated by using an example in which the annular rotating frame 200 in the rotating gantry 000 is disk-shaped. In other optional embodiments, the structure of the drive assembly shown in the first optional embodiment may also be applied to a rotating gantry provided with a roll-shaped annular rotating frame. In an exemplary embodiment, referring to FIG. 11, FIG. 11 is a schematic structural view of another rotating gantry according to another embodiment of the present disclosure. For the structure of the drive assembly 300 in the rotating gantry 000, reference may be made to the structure shown in the first optional embodiment. Details are not repeated in this embodiment of the present disclosure.

It should be noted that, as seen from the structure of the rotating gantry shown in FIG. 11, the rotating gantry 000 may include two drive assemblies 300. By the two drive assemblies 300, the roller-shaped annular rotating frame 200 having a large mass can be easily driven to rotate. The rotating gantry 000 may further include an auxiliary drive assembly 600. The auxiliary drive assembly 600 may include an auxiliary drive gear 601 meshed with the drive gear 302 and a handle (not show in FIG. 11) detachably connected to the auxiliary drive gear 601. Due to the relatively large mass of the roller-shaped annular rotating frame 200 in the rotating gantry 000, in the case that the drive assembly 300 is powered down, it is hard for an operator to rotate the annular rotating frame 200. In this case, the operator can easily operate and control, via the handle and the auxiliary drive gear 601, the annular rotating frame 200 to rotate.

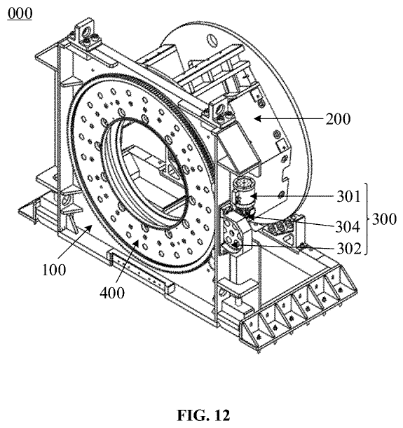

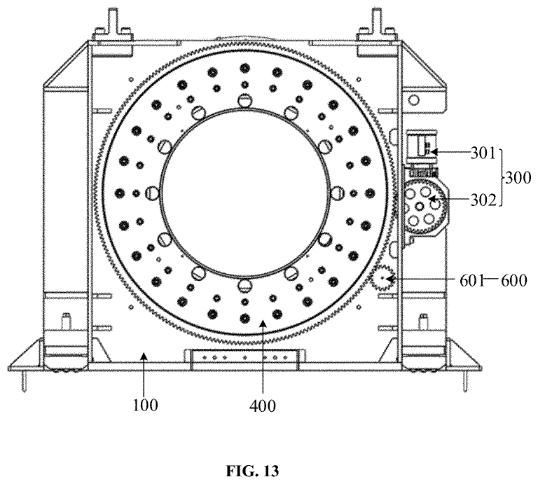

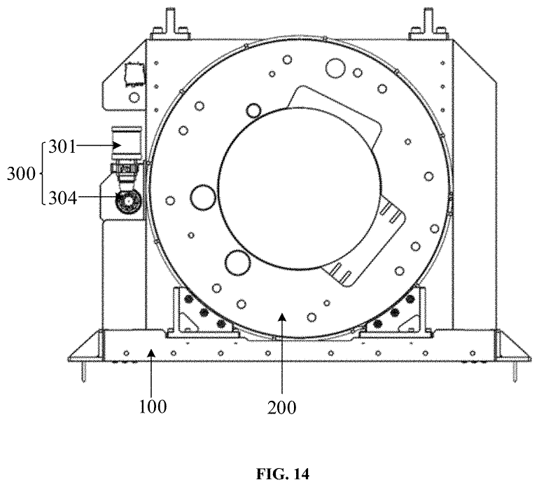

In a second optional embodiment, as shown in FIG. 12, FIG. 13, and FIG. 14, FIG. 12 is a schematic structural view of still another rotating gantry according to another embodiment of the present disclosure, FIG. 13 is a schematic view of a plane on one side of the rotating gantry shown in FIG. 12, and FIG. 14 is a schematic view of a plane on the other side of the rotating gantry shown in FIG. 12. The drive assembly 300 in the rotating gantry 000 may further include a right-angle reducer 304 fixedly connected to the frame-shaped support 100. The right-angle reducer 304 may be disposed between the drive motor 301 and the drive gear 302 in the drive assembly 300. The drive motor 301 may be connected to the drive gear 302 via the right-angle reducer 304.

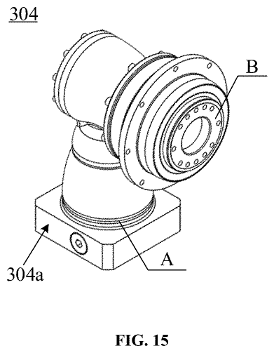

In an embodiment, referring to FIG. 15, FIG. 15 is a schematic structural view of a right-angle reducer according to an embodiment of the present disclosure. The drive motor 301 in the drive assembly 300 is provided with a transmission shaft. The transmission shaft of the drive motor 301 may be connected to an end A of the right-angle reducer 304; and the other end B of the right-angle reducer 304 may be connected to the drive gear 302. The axis of the transmission shaft of the drive motor 301 may be perpendicular to the axis of the drive gear 302. The right-angle reducer 304 is provided with a support plate 304a. The right-angle reducer 304 may be fixedly connected to the frame-shaped support 100 via the support plate 304a.

In this case, due to the connection via the right-angle reducer 304, the drive motor 301 can be disposed outside the frame-shaped support 100, such that the positions of the components in the rotating gantry 000 are closer. In addition, in the case that the drive motor 301 is disposed outside the frame-shaped support 100, a high-power drive motor 301 can be selected on the premise that the entire volume of the rotating gantry 000 is not affected, which facilitates driving the annular rotating frame 200 to rotate.

In some embodiments, as shown in FIG. 13, the rotating gantry 000 may further include an auxiliary drive assembly 600. The auxiliary drive assembly 600 may include an auxiliary drive gear 601 meshed with the ring gear disk 400 and a handle (not show in FIG. 13) detachably connected to the auxiliary drive gear 601. For the function and operating principle of the auxiliary drive assembly 600, reference may be made to corresponding content in the above embodiments. Details are not repeated in the present disclosure.

It should be noted that, FIG. 12 is illustrated by using an example in which the annular rotating frame 200 in the rotating gantry 000 is roller-shaped. In other optional embodiments, the structure of the drive assembly shown in the second optional embodiment may also be applied to a rotating gantry provided with a disk-shaped annular rotating frame. This is not limited in this embodiment of the present disclosure.

In summary, the rotating gantry in this embodiment of the present disclosure includes a frame-shaped support, an annular rotating frame, a drive assembly, and a ring gear disk. The drive assembly is configured to drive, via the ring gear disk, the annular rotating frame to rotate. The annular rotating frame in the rotating gantry is disposed on one side of the frame-shaped support; and the ring gear disk and the drive assembly are disposed on the other side of the frame-shaped support. In this case, components in the rotating gantry are arranged closely. In addition, the frame-shaped support takes the shape of a flat plate. Therefore, the size of the rotating gantry is relatively small, which effectively reduces the volume of the rotating gantry. In this way, the transport difficulty of the rotating gantry can be effectively lowered. In addition, the frame-shaped support can be directly disposed on the ground, instead of being pre-embedded in the ground, which effectively reduces the mounting difficulty of the rotating gantry.

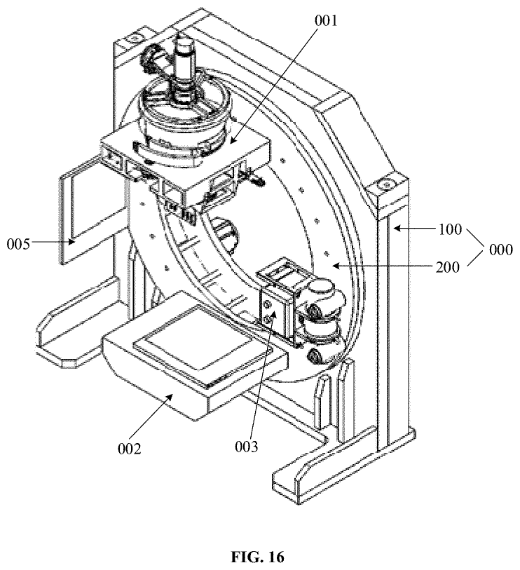

Referring to FIG. 16, FIG. 16 is a schematic structural view of radiotherapy equipment according to an embodiment of the present disclosure. The radiotherapy equipment may include a rotating gantry 000 and a target device. The rotating gantry 000 may be the rotating gantry 000 shown in the above embodiments. The target device may be connected to the annular rotating frame 200 in the rotating gantry 000.

In some embodiments, the target device in the radiotherapy equipment may include a treatment head 001 and a shield 002 that are arranged oppositely. In the case that the annular rotating frame 200 in the rotating gantry 000 is disk-shaped, the treatment head 001 and the shield 002 may be both fixedly connected to an end surface on one side, distal from the frame-shaped support 100, of the annular rotating frame 200. The radiotherapy equipment is provided with a treatment area. The treatment area is disposed between the treatment head 001 and the shield 002. The shield 002 can shield a radiation beam that is emitted by the treatment head 001 and passes through the treatment area. Therefore, the shield requirement of a machine room in a hospital can be lowered by the shield 002, thereby effectively reducing the reconstruction cost for an original machine room.

In this embodiment of the present disclosure, the target device in the radiotherapy equipment may further include an imaging assembly 003 and a detector 005 that are arranged oppositely. The treatment area of the radiotherapy equipment may also be disposed between the imaging assembly 003 and the detector 005. A direction of a radiation beam emitted by the imaging assembly 003 may be intersected with a direction of a radiation beam emitted by the treatment head 001. For example, the direction of the radiation beam emitted by the imaging assembly 003 may be perpendicular to the direction of the radiation beam emitted by the treatment head 001. The radiation beam, emitted by the imaging assembly 003 and passing through a target area of a patient in the treatment area, is incident to the detector 005. The detector 005 performs collection and imaging based on the radiation beam, such that the radiotherapy equipment can acquire an image of the target area of the patient in real-time.

In this embodiment of the present disclosure, the radiotherapy equipment may further include an auxiliary device configured to supply power and dissipate heat for the treatment head 001, the imaging assembly 003, and the detector 005. The auxiliary device may also be fixedly connected to the end surface on one side, distal from the frame-shaped support 100, of the annular rotating frame 200. In this case, most of the components of the radiotherapy equipment are integrated on an end surface of the annular rotating frame 200, such that the components of the radiotherapy equipment are arranged more closely, which effectively reduces the volume of the radiotherapy equipment. In addition, the radiotherapy equipment can be wholly transported; and components of the radiotherapy equipment can be maintained and installed more conveniently.

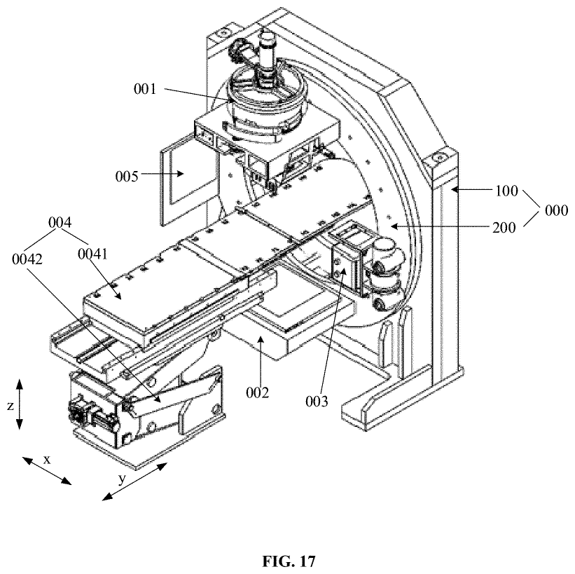

In some embodiments, referring to FIG. 7, FIG. 17 is a schematic structural view of another radiotherapy equipment according to an embodiment of the present disclosure. The radiotherapy equipment may further include a treatment couch 004. The treatment couch 004 may be disposed on one side, proximal to the annular rotating frame 200, of the rotating gantry 000 of the radiotherapy equipment. In an exemplary embodiment, the treatment couch 004 may include a treatment couch body 0041 and a foldable support frame 0042 fixedly connected to the treatment couch body 0041. The foldable support frame 0042 is configured to drive the treatment couch body 0041 to move in the height direction z of the radiotherapy equipment.

In this embodiment of the present disclosure, the foldable support frame 0042 can drive, by folding, the treatment couch body 0041 to move to a position closer to the ground. In this way, a patient can get onto the couch easily with no need to embed a treatment couch or a rotating gantry couch into a pit, thereby further reducing the mounting difficulty of the radiotherapy equipment.

It should be noted that, the treatment couch 004 in this embodiment of the present disclosure can further drive the treatment couch body 0041 to move in the horizontal direction x and the vertical direction v of the treatment couch 004.

In this embodiment of the present disclosure, the inner diameter of the annular rotating frame 200 is greater than or equal to 1 meter. In this way, the treatment area of the radiotherapy equipment can meet the treatment requirement.

In the embodiments of the present disclosure, the terms “first” and “second” are used only for descriptive purposes and cannot be construed as indicating or implying relative importance. Unless otherwise specified, the term “a plurality of” means two or more.

Described above are merely optional embodiments of the present disclosure, but are not intended to limit the present disclosure. Any modifications, equivalent replacements, improvements and the like made within the concepts and principles of the present disclosure should be included within the scope of protection of the present disclosure.

What is claimed is:

1. A rotating gantry, comprising:
a frame-shaped support;
an annular rotating frame disposed on one side of the frame-shaped support and configured to support a target device;
a ring gear disk disposed on the other side of the frame-shaped support and fixedly connected to the annular rotating frame; and
a drive assembly comprising a drive motor and a drive gear, wherein the drive motor is connected to the drive gear, the drive gear is meshed with the ring gear disk, and under driving of the drive motor, the drive gear is configured to drive the ring gear disk and the annular rotating frame to rotate relative to the frame-shaped support, such that the target device is driven to rotate.

2. The rotating gantry according to claim 1, wherein the drive motor is provided with a transmission shaft, wherein the drive gear is sleeved on the transmission shaft and is fixedly connected to the transmission shaft.

3. The rotating gantry according to claim 2, wherein the drive motor is a torque motor.

4. The rotating gantry according to claim 2, wherein the drive assembly further comprises a mounting base, wherein the mounting base is fixedly connected to one side, distal from the annular rotating frame, of the frame-shaped support, and is fixedly connected to the drive motor and movably connected to the drive gear.

5. The rotating gantry according to claim 4, wherein an axis of the mounting base is not coincident with an axis of the drive gear.

6. The rotating gantry according to claim 5, wherein:
the mounting base comprises a base body and a positioning member, wherein the positioning member is fixedly connected to one side of the base body, the other side of the base body is fixedly connected to the drive motor, and the base body is movably connected to the drive gear;
one side, distal from the annular rotating frame, of the frame-shaped support is provided with a positioning hole, wherein the positioning hole is matched with the positioning member in shape, and in the case that the positioning member is disposed in the positioning hole and the drive gear is meshed with the ring gear disk, one side, distal from the drive motor, of the base body is fixedly connected to one side, distal from the annular rotating frame, of the frame-shaped support

7. The rotating gantry according to claim 6, wherein:
the base body is provided with a receiving chamber, wherein an outer side surface of the base body is provided with an opening communicated with the receiving chamber, the drive gear is disposed in the receiving chamber, and the drive gear is meshed with the ring gear disk through the opening; and
the mounting base further comprises a support bearing disposed between the positioning member and the transmission shaft, wherein the positioning member is movably connected to the transmission shaft via the support bearing.

8. The rotating gantry according to claim 1, wherein the drive assembly further comprises a right-angle reducer fixedly connected to the frame-shaped support, wherein the right-angle reducer is disposed between the drive motor and the drive gear, and the drive motor is connected to the drive gear via the right-angle reducer.

9. The rotating gantry according to claim 8, wherein the drive motor is provided with a transmission shaft, wherein the transmission shaft is connected to one end of the right-angle reducer, the other end of the right-angle reducer is connected to the drive gear, and an axis of the transmission shaft is perpendicular to an axis of the drive gear.

10. The rotating gantry according to claim 1, further comprising a rotary assembly, wherein the rotary assembly is disposed on the frame-shaped support between the annular rotating frame and the ring gear disk, and the ring gear disk and the annular rotating frame are movably connected to the frame-shaped support via the rotary assembly.

11. The rotating gantry according to claim 10, wherein the rotary assembly further comprises an inner ring body and an outer ring body movably connected to the inner ring body, wherein the outer ring body is fixedly connected to the frame-shaped support, and the inner ring body is fixedly connected to the annular rotating frame and the ring gear disk separately.

12. The rotating gantry according to claim 11, wherein the rotary assembly further comprises a plurality of spheres disposed between the inner ring body and the outer ring body, wherein the inner ring body is movably connected to the outer ring body via the plurality of spheres.

13. The rotating gantry according to claim 1, wherein the annular rotating frame is disk-shaped, and the target device is fixedly connected to one side, distal from the frame-shaped support, of the disk-shaped annular rotating frame.

14. The rotating gantry according to claim 1, wherein the annular rotating frame is roller-shaped, and the target device is fixedly connected to an inner wall of the roller-shaped annular rotating frame.

15. The rotating gantry according to claim 1, wherein the ring gear disk and the drive gear are both an external gear.

16. A radiotherapy equipment, comprising:
a rotating gantry and a target device, wherein the rotating gantry comprises:
a frame-shaped support;
an annular rotating frame disposed on one side of the frame-shaped support and connected to the target device;
a ring gear disk disposed on the other side of the frame-shaped support and fixedly connected to the annular rotating frame; and
a drive assembly comprising a drive motor and a drive gear, wherein the drive motor is connected to the drive gear, the drive gear is meshed with the ring gear disk, and under driving of the drive motor, the drive gear is configured to drive the ring gear disk and the annular rotating frame to rotate relative to the frame-shaped support, such that the target device is driven to rotate.

17. The radiotherapy equipment according to claim 16, wherein the target device comprises a treatment head and a shield that are arranged oppositely; and the radiotherapy equipment is provided with a treatment area, wherein the treatment area is disposed between the treatment head and the shield.

18. The radiotherapy equipment according to claim 17, wherein the target device further comprises an imaging assembly and a detector that are arranged oppositely, wherein the treatment area is disposed between the imaging assembly and the detector, and a direction of a radiation beam emitted by the imaging assembly is intersected with a direction of a radiation beam emitted by the treatment head.

19. The radiotherapy equipment according to claim 16, further comprising a treatment couch, wherein the treatment couch is disposed on one side, proximal to the annular rotating frame, of the rotating gantry.

20. The radiotherapy equipment according to claim 19, wherein the treatment couch comprises a treatment couch body and a foldable support frame fixedly connected to the treatment couch body, wherein the foldable support frame is configured to drive the treatment couch body to move in a height direction of the radiotherapy equipment.
